(19) United States
(12) Patent Application Publication
(10) Pub. No.: US 2021/0100428 A1
(43) Pub. Date: Apr. 8, 2021

(54) DEVICES, SYSTEMS, AND METHODS FOR POSITIONING A MEDICAL DEVICE WITHIN A BODY LUMEN

(71) Applicant: Boston Scientific Scimed, Inc., Maple Grove, MN (US)

(72) Inventors: Oscar R. Carrillo, JR., Middletown, CT (US); Barry Weitzner, Acton, MA (US); Daniel J. Foster, Lino Lakes, MN (US); Megan Chrobak, Groton, MA (US); Mark W. Boden, Harrisville, RI (US); Elizabeth M. Albrecht, White Bear Lake, MN (US)

(21) Appl. No.: 17/039,173

(22) Filed: Sep. 30, 2020

Related U.S. Application Data
(60) Provisional application No. 62/911,772, filed on Oct. 7, 2019.

(51) Int. Cl. A61B 1/005; A61B 1/01; A61B 1/00

(52) U.S. Cl. CPC A61B 1/0052; A61B 1/01; A61B 1/00006; A61B 1/00045; A61B 1/00039

(57) ABSTRACT

Various embodiments are generally directed to positioning techniques to facilitate entry of a flexible elongate member (e.g., endoscopic accessory tool) into and/or through selected anatomies, such as by directing a set of positional controls based on feedback from a set of transducers to achieve or maintain a position within a body lumen, for instance. Some embodiments are particularly directed to a feedback loop for positioning or guiding a flexible elongate member into a target configuration for one or more of inspection, orientation, and/or facilitating access to body passageways. In one embodiment, for example, a device manager may include circuitry to operate at least one positional control in a set of positional controls based on data indicative of a current configuration of a flexible elongate member generated by a set of transducers.

### CROSS-REFERENCE TO RELATED APPLICATION

The present application claims the benefit of priority under 35 U.S.C. § 119 to U.S. Provisional Patent Application 62/911,772, filed Oct. 7, 2019, which application is incorporated herein by reference in its entirety for all purposes.

### FIELD

The present disclosure relates generally to the field of medical devices. In particular, the present disclosure relates to devices, systems, and methods to facilitate entry of a flexible elongate member into and/or through selected anatomies.

### BACKGROUND

Generally, performing endoscopic cannulation procedures requires advancing a guidewire and/or endoscopic accessory tool (e.g., sphincterotome, cannula, catheter, transducer, etc.) into and through patient anatomies. An exemplary endoscopic cannulation procedure may include an Endoscopic Retrograde Cholangio-Pancreatography (ERCP) procedure to access and examine the biliary duct. During such an ERCP procedure, an endoscope is inserted through the mouth and advanced to the duodenum. An attempt is made to identify the common entry point for the biliary and pancreatic ducts. Once successfully identified, a guidewire may be advanced into the biliary duct to perform a variety of therapeutic procedures, such as stone management or therapy of biliary malignancies. Multiple attempts to access the biliary duct may result in a prolonged or failed procedure. In addition, tissue trauma may result from the multiple access attempts.

It is with these considerations in mind that a variety of advantageous medical outcomes may be realized by the devices, systems, and methods of the present disclosure.

### SUMMARY

In one aspect, the present disclosure relates to a medical device comprising a flexible elongate member having a proximal end, a distal end, and a length therebetween. A set of one or more positional controls may be disposed along the length of the flexible elongate member. Each positional control in the set of one or more positional controls may be configured to adjust at least a portion of the flexible elongate member. The set of one or more positional controls may include a joint, an actuator, a motor, a balloon, a guidewire, or some combination thereof. In various embodiments, at least a portion of the set of one or more positional controls may adjust one or more of a yaw, a pitch, and a roll of the distal end of the flexible elongate member. In many embodiments, at least one position control in the set of one or more positional controls configured to contact a wall of a body lumen to adjust a location of one or more portions of the flexible elongate member within the body lumen. A set of one or more transducers may be disposed along the length of the flexible elongate member. At least a portion of the set of one or more positional controls may be configured to generate data indicative of a configuration of the flexible elongate member. The set of one or more transducers may include an optical sensor, a range finder, an accelerometer, a gyroscope, a pressure sensor, a temperature sensor, a force sensor, or an electromagnetic sensor, or some combination thereof. In some embodiments, at least a portion of the set of one or more transducers may be configured to monitor an environment of at least a portion of the flexible elongate member. In some such embodiments, the environment of at least a portion of the flexible elongate member may comprise an interior of a body lumen. In various embodiments, the medical device may include a device manager comprising circuitry. The circuitry may be configured to operate at least one positional control in the set of one or more positional controls based on data indicative of the configuration of the flexible elongate member generated by the set of one or more transducers. The circuitry may be configured to monitor a position of the flexible elongate member within a body lumen based on the data generated by the set of one or more transducers. In some embodiments, the medical device may include a user interface communicatively coupled to the device manager. In some such embodiments, the device manager may include circuitry configured to operate at least one positional control in the set of one or more positional controls based on input received via the user interface. In various embodiments, the device manager may include circuitry configured to display the configuration of the flexible elongate member via the user interface. In one or more embodiments, the circuitry of the device manager may include a processor and memory. In several embodiments, the circuitry of the device manager may be configured to determine a suggested position of at least a portion of the flexible elongate member. In several such embodiments, the circuitry of the device manager may be configured to communicate the suggested position of at least a portion of the flexible elongate member via the user interface.

In another aspect, the present disclosure relates to an apparatus comprising a processor and a memory comprising instructions that when executed by the processor cause the processor to perform one or more of the following. The memory may include instructions to cause the processor to identify one or more feedback signals generated by a set of one or more transducers disposed along a length of a flexible elongate member. The memory may include instructions to cause the processor to determine a current configuration of at least one portion of the flexible elongate member within a body lumen based on one or more portions of the one or more feedback signals generated by the set of one or more transducers. The memory may include instructions to cause the processor to compare the current configuration of the at least one portion of the flexible elongate member to a target configuration of the at least one portion of the flexible elongate member. In some embodiments, the memory may include instructions to cause the processor to communicate the target configuration of the at least one portion of the flexible elongate member via a user interface. In various embodiments, the memory may include instructions to cause the processor to communicate the current configuration of the at least one portion of the flexible elongate member via a user interface. In many embodiments, the memory may include instructions to cause the processor to determine the target configuration of the at least one portion of the flexible elongate member based on a previous configuration of the at least one portion of the flexible elongate member. In several embodiments, the memory may include instructions to cause the processor to determine the target configuration of the at least one portion of the flexible elongate member based on input received via a user interface. The memory may include instructions to cause the processor to generate one or more control signals to operate at least one positional control in a set of one or more positional controls disposed along the length of the flexible elongate member to adjust the flexible elongate based on comparison of the current configuration of the at least one portion of the flexible elongate member to the target configuration. In many embodiments, the memory may include instructions to cause the processor to determine an environment of the at least one portion of the flexible elongate member within the body lumen based on at least one portion of the one or more feedback signals generated by the set of one or more transducers. In several embodiments, the memory may include instructions to cause the processor to operate at least one positional control in the set of one or more positional controls based on input received via a user interface.

In yet another aspect, the present disclosure relates to a method. The method may include identifying one or more feedback signals generated by a set of one or more transducers disposed along a length of a flexible elongate member. The method may include determining a current configuration of at least one portion of the flexible elongate member within a body lumen based on one or more portions of the one or more feedback signals generated by the set of one or more transducers. The method may include comparing the current configuration of the at least one portion of the flexible elongate member to a target configuration of the at least one portion of the flexible elongate member. In various embodiments, the method may include communicating the target configuration of the at least one portion of the flexible elongate member via a user interface. In some embodiments, the method may include communicating the current configuration of the at least one portion of the flexible elongate member via a user interface. The method may include generating one or more control signals to operate at least one positional control in a set of one or more positional controls disposed along the length of the flexible elongate member to adjust the flexible elongate based on comparison of the current configuration of the at least one portion of the flexible elongate member to the target configuration. In many embodiments, the method may include determining an environment of the at least one portion of the flexible elongate member within the body lumen based on at least one portion of the one or more feedback signals generated by the set of one or more transducers. In some embodiments, the method may include operating at least one positional control in the set of one or more positional controls based on input received via a user interface. In several embodiments, the method may include determining the target configuration of the at least one portion of the flexible elongate member based on a previous configuration of at least one portion of the flexible elongate member.

### BRIEF DESCRIPTION OF THE DRAWINGS

Non-limiting embodiments of the present disclosure are described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment shown where illustration is not necessary to allow those of ordinary skill in the art to understand the disclosure. In the figures:

FIG. 1 illustrates examples of aspects of a medical device, according to one or more embodiments described herein.

FIG. 2 illustrates an example of an operating environment for a flexible elongate member of a medical device, according to one or more embodiments described herein.

FIG. 3 illustrates an example of a process flow for a medical device, according to one or more embodiments described herein.

FIG. 4 illustrates various aspects of positioning a flexible elongate member in an example of an operating environment, according to one or more embodiments described herein.

FIG. 5 illustrates various aspects of positioning a flexible elongate member in an example of an operating environment, according to one or more embodiments described herein.

FIG. 6 illustrates various aspects of positioning a flexible elongate member in an example of an operating environment, according to one or more embodiments described herein.

FIG. 7 illustrates examples of aspects of a computing architecture, according to one or more embodiments described herein.

### DETAILED DESCRIPTION

Various embodiments are generally directed to positioning techniques to facilitate entry of a flexible elongate member (e.g., endoscopic accessory tool) into and/or through anatomies, such as by directing a set of positional controls based on feedback from a set of transducers to achieve or maintain a position within a body lumen, for instance. Some embodiments are directed to a feedback loop for positioning or guiding a flexible elongate member into a target configuration for one or more of inspection, orientation, and/or facilitating access to body passageways. In one embodiment, for example, a flexible elongate member may include a set of transducers and a set of positional controls disposed along the length of the flexible elongate member. In such embodiments, a device manager may include circuitry to operate at least one positional control in the set of positional controls based on data indicative of a current configuration of a flexible elongate member generated by the set of transducers.

In various embodiments, the circuitry of the device manager may include a processor and memory with executable instructions to cause the processor to perform one or more of the following operations. In some embodiments, the processor may identify feedback signals generated by a set of transducers disposed along the length of a flexible elongate member. In some such embodiments, the processor may determine a current configuration of the flexible elongate member based on the feedback signals. In embodiments, the processor may compare the current configuration of the flexible elongate member to a target configuration. In embodiments, the processor may operate a set of positional controls disposed along the length of the flexible elongate member to maneuver the flexible elongate member into the target configuration. These and other embodiments are described and claimed.

Some challenges in facilitating entry of a flexible elongate member into and/or through selected anatomies include locating a selected anatomy and positioning a distal end of the flexible elongate member appropriately to access the selected anatomy. Such challenges may result from several factors, such as the ergonomics of manipulating a multiple degrees of freedom (e.g., eight) endoscope, such as a duodenoscope, to a precise location and the inability to visualize obscured or hidden entry points. For example, a target body passageway may be oriented at a difficult angle relative to an endoscopic accessory tool (e.g., obtuse angles, orthogonal, oblique), have a very small or sealed opening, or include a tortuous anatomy, blockages (e.g., stones, etc.) and benign or malignant structures. Medical professionals may make multiple attempts to achieve successful cannulations. Further, the likelihood of causing trauma to the tissues comprising or surrounding the target body passageway increases with the number of cannulation attempts.

For example, the inability to cannulate the common bile duct is one reason for a failed ERCP procedure. Adding further complexity, during the cannulation process, information regarding selected, anatomy-associated procedural objectives may be unavailable. For instance, positional/orientation information regarding the anatomy of the ducts beyond the entry point(s) may be unavailable. Without information regarding the anatomy of the ducts, medical professionals attempt to maneuver a guidewire blindly into the biliary duct. One or more selected anatomies described herein may include a patient-specific anatomy. For example, in some patients, the entry point may be a common entrance to the biliary and pancreatic ducts in the duodenal wall and in other patients, the biliary and pancreatic ducts may have separate entry points in the duodenal wall.

Various embodiments described herein include medical devices capable of configuring a flexible elongate member for access to the selected anatomy and/or accessing the selected anatomy in a safe, accurate, and reliable manner. In embodiments, one or more devices described herein may utilize one or more of a user interface, a set of transducers, a set of positional controls, and a device manager to monitor and/or control the position of a flexible elongate member (e.g., device for cannulation procedure) inside an anatomy in a reliable and proficient manner with limited or no user input. In embodiments, the device manager may guide one or more aspects of cannulation procedures based on data generated by a set of transducers disposed along the length of the flexible elongate member. For example, one or more of a current and a target configuration of the flexible elongate member may be presented via the user interface. In some examples, the target configuration may be determined by the device manager based on one or more of a previous configuration of the flexible elongate member and user input.

In various embodiments, the device manager may control one or more aspects of cannulation procedures via a set of positional controls disposed along the length of the flexible elongate member. For instance, the device manager may position the flexible elongate member into a target configuration (e.g., a suggested position of at least a portion of the flexible elongate member). In such instances, the device manager may position the flexible elongate member into the target configuration based on one or more of a current configuration, a target configuration, and user input.

In many embodiments, the flexible elongate member may be navigated to the appropriate location and locked in place for cannulation and/or visualization during a cannulation procedure. In some embodiments, the medical device may automatically maintain the flexible elongate member in position, such as by countering back pressure or movement caused by tools inserted via a working channel of the flexible elongate member and utilized during a cannulation procedure. In embodiments, this may avoid the need for repositioning and reduce complications. Oftentimes, one or more medical devices described herein may remove the need for a medical professional to manually adapt to patient movements during cannulation procedures.

In these and other ways, components/techniques described here may improve patient care, increase user experience, decrease learning curves, improve success rates, and/or decrease adverse outcomes via realization of a more efficient and better functioning medical device with advantageous features. In many embodiments, one or more of the advantageous features may result in several technical effects and advantages over conventional computer technology, including increased capabilities and improved adaptability. In various embodiments, one or more of the aspects, techniques, and/or components described herein may be implemented in a practical application via one or more computing devices, and thereby provide additional and useful functionality to the one or more computing devices, resulting in more capable, better functioning, and improved computing devices. Further, one or more of the aspects, techniques, and/or components described herein may be utilized to improve one or more technical fields including cannulation, diagnosis, treatment, robotics, embedded systems, user experience, and/or control systems.

In several embodiments, components described herein may provide specific and particular manners to enable position detection and/or control during cannulation procedures. In several such embodiments, the specific and particular manners may include, for instance, controlling, monitoring, and/or interfacing with one or more of a flexible elongate member, a transducer, a positional control, and a user interface to facilitate one or more cannulation procedures. In one example, the specific and particular manner may simplify ERCP procedures, such as by automating one or more aspects, to enable medical professional to quickly learn to safely and reliably access the biliary duct.

In embodiments, one or more of the components described herein may be implemented as a set of rules that improve computer-related technology by allowing a function not previously performable by a computer that enables an improved technological result to be achieved. In embodiments, the function allowed may be associated with cannulation devices and/or procedures. For example, the function allowed may include directing a set of positional controls disposed along the length of a flexible elongate member based on feedback from a set of transducers disposed along the length of the flexible elongate member to achieve and/or maintain a position within a body lumen. In some embodiments, the function allowed may include determining a current and/or target configuration for a flexible elongate member to achieve and/or maintain a position within a body lumen. In some such embodiments, that target configuration may be determined based on feedback from the set of transducers and/or input received via the user interface. In various embodiments, the function allowed may include locating and/or accessing one or more objectives of a cannulation procedure.

The present disclosure is not limited to the particular embodiments described. The terminology used herein is for the purpose of describing particular embodiments only and is not intended to be limiting beyond the scope of the appended claims. Unless otherwise defined, all technical terms used herein have the same meaning as commonly understood by one of ordinary skill in the art to which the disclosure belongs.

Although embodiments of the present disclosure may be described with specific reference to medical devices and systems (e.g., endoscopic accessory tools and/or guidewires inserted through a duodenoscope, etc.) for selective cannulation of the common bile duct (CBD) or pancreatic duct (PD) during an Endoscopic Retrograde Cholangio-Pancreatography (ERCP) procedure, it should be appreciated that such medical devices and systems may be used in a variety of medical procedures which require navigating one or more accessory tools through ductal, luminal, or vascular anatomies, including, for example, interventional radiology procedures, balloon angioplasty procedures, thrombolysis procedures, angiography procedures and the like. The medical devices of the present disclosure are not limited to duodenoscopes, and may include a variety of medical devices for accessing body passageways, including, for example, catheters, ureteroscopes, bronchoscopes, colonoscopes, arthroscopes, cystoscopes, hysteroscopes, and the like. Further, the disclosed medical devices and systems may be inserted via different access points and approaches, e.g., percutaneously, endoscopically, laparoscopically or some combination thereof.

As used herein, the singular forms “a,” “an,” and “the” are intended to include the plural forms as well, unless the context clearly indicates otherwise. It will be further understood that the terms “comprises” and/or “comprising,” or “includes” and/or “including” when used herein, specify the presence of stated features, regions, steps, elements and/or components, but do not preclude the presence or addition of one or more other features, regions, integers, steps, operations, elements, components and/or groups thereof.

As used herein, the term “distal” refers to the end farthest away from the medical professional when introducing a device into a patient, while the term “proximal” refers to the end closest to the medical professional when introducing a device into a patient.

With general reference to notations and nomenclature used herein, one or more portions of the detailed description which follows may be presented in terms of program procedures executed on a computer or network of computers. These procedural descriptions and representations are used by those skilled in the art to most effectively convey the substances of their work to others skilled in the art. A procedure is here, and generally, conceived to be a self-consistent sequence of operations leading to a desired result. These operations are those requiring physical manipulations of physical quantities. Usually, though not necessarily, these quantities take the form of electrical, magnetic, or optical signals capable of being stored, transferred, combined, compared, and otherwise manipulated. It proves convenient at times, principally for reasons of common usage, to refer to these signals as bits, values, elements, symbols, characters, terms, numbers, or the like. It should be noted, however, that all of these and similar terms are to be associated with the appropriate physical quantities and are merely convenient labels applied to those quantities.

Further, these manipulations are often referred to in terms, such as adding or comparing, which are commonly associated with mental operations performed by a human operator. However, no such capability of a human operator is necessary, or desirable in most cases, in any of the operations described herein that form part of one or more embodiments. Rather, these operations are machine operations. Useful machines for performing operations of various embodiments include general purpose digital computers as selectively activated or configured by a computer program stored within that is written in accordance with the teachings herein, and/or include apparatus specially constructed for the required purpose. Various embodiments also relate to apparatus or systems for performing these operations. These apparatuses may be specially constructed for the required purpose or may include a general-purpose computer. The required structure for a variety of these machines will be apparent from the description given.

Reference is now made to the drawings, wherein like reference numerals are used to refer to like elements throughout. In the following description, for purpose of explanation, numerous specific details are set forth in order to provide a thorough understanding thereof. It may be evident, however, that the novel embodiments can be practiced without these specific details. In other instances, well known structures and devices are shown in block diagram form to facilitate a description thereof. The intention is to cover all modification, equivalents, and alternatives within the scope of the claims.

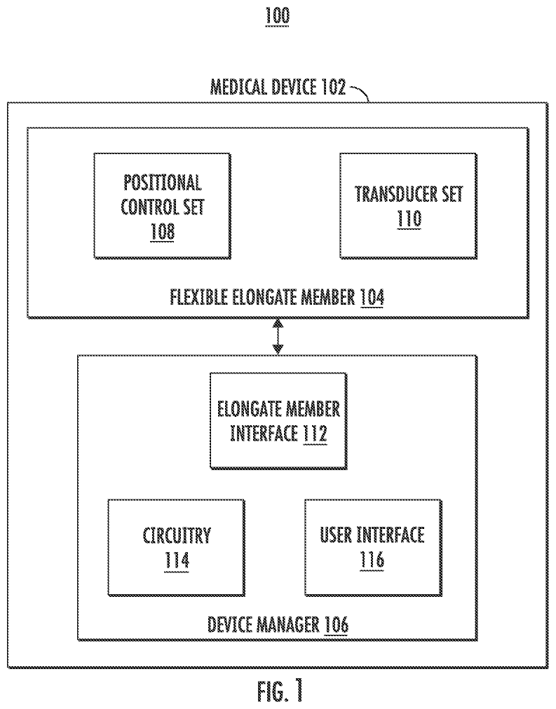

FIG. 1 illustrates an example of an aspect of medical device 102 in operating environment 100 according to one or more embodiments described herein. In operating environment 100, medical device 102 may include a flexible elongate member 104 and a device manager 106. The flexible elongate member 104 includes a positional control set 108 and a transducer set 110. The device manager 106 includes an elongate member interface 112, circuitry 114, and a user interface 116. In one or more embodiments described herein, the components of medical device 102 may interoperate to facilitate cannulation procedures within a body lumen, such as by operating one or more positional controls in positional control set 108 based on feedback signals generated by one or more transducers in transducer set 110. Embodiments are not limited in this context.

In embodiments, device manager 106 may manage, monitor, and/or operate one or more components of flexible elongate member 104. In many such embodiments, device manager 106 may manage, monitor, and/or operate one or more components of flexible elongate member based on input/output received/generated by circuitry 114. For example, circuitry 114 may include a processor and memory with instructions to cause the processor to implement one or more techniques described herein. In one or more embodiments, circuitry 114 may communicate information associated with, or generated by, one or more components of the flexible elongate member 104. In one or more such embodiments, circuitry 114 may enable monitoring or controlling one or more components of the flexible elongate memory 104 based on input/output received/sent via elongate member interface 112 and/or user interface 116.

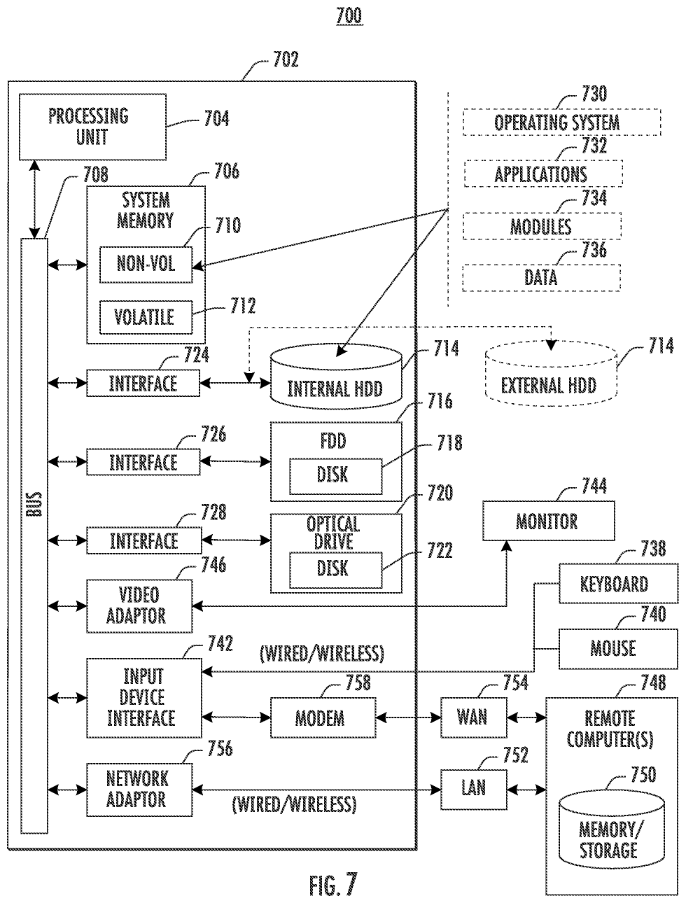

For example, device manager 106 may maintain flexible elongate member 104 in a target configuration (e.g., position, orientation, mode, etcetera) identified, at least in part, via user interface 116. In such examples, device manager 106 may maintain flexible elongate member 104 in the target configuration via positional control set 108 and/or transducer set 110. In various embodiments, one or more components of device manager 106 may be the same or similar to one or more components illustrated and described with respect to FIG. 7. In various such embodiments, device manager 106 may additionally, or alternatively, include one or more components illustrated and described with respect to FIG. 7.

In various embodiments, motion of the flexible elongate member 104 (e.g., in and out, rotation, up and down, left and right, elevator controls, and the like) may be integrated into feedback system/drive mechanism implemented in or controlled by device manager 106 (e.g., via circuitry 114). In many embodiments, medical device 102 may be implemented on, or comprise, a cable and/or robotic driven scope. In several embodiments, flexible elongate member 104 may include one or more vertebrae that can be independently driven, such as by positional controls in positional control set 108.

In one or more embodiments, the components of medical device 102 may interoperate to adjust the flexible elongate member 104 to be in a target position, such as a preset position determined by input received via user interface 116. In several embodiments, device manager 106 may localize and/or detect disturbances to a location of the flexible elongate member using sensors (e.g., included in transducer set 110). In several such embodiments, device manager 106 may counter and/or correct disturbances to the position of flexible elongate member 104 using one or more positional controls in positional control set 108.

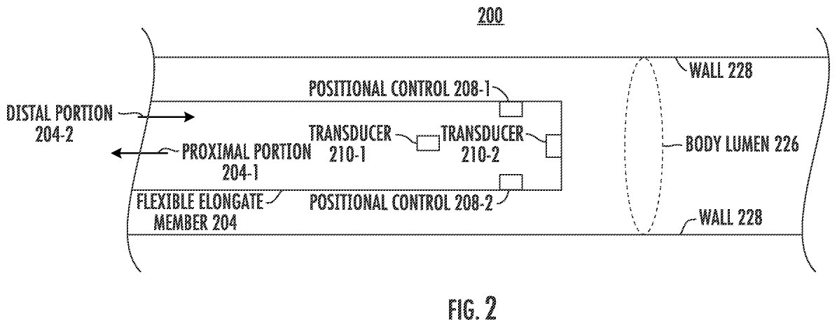

In some embodiments, the flexible elongate member 104 may be used as a stand-alone device for insertion into a body lumen during a cannulation procedure. However, in additional, or alternative, embodiments the flexible elongate member 104 may be configured to extend through the working channel of another medical device (e.g., a duodenoscope, ureteroscope, bronchoscope, colonoscope, arthroscope, cystoscope, hysteroscope, other type of endoscope, catheter, introducer sheath, catheter, or the like, etc.). In various embodiments, flexible elongate member 104 may include a proximal portion/region with a proximal end and a distal portion/region with a distal end. In various such embodiments, the distal region may be inserted into a body lumen. In one or more embodiments, the flexible elongate member 104 may include at least a portion of an endoscope, an endoscopic accessory tool, a tome, a cannula, a catheter, and the like. As will be described in more detail below, such as with respect to FIG. 2, in many embodiments, positional control set 108 and the transducer set 110 may be disposed along the length of the flexible elongate member 104 between the proximal and distal ends. In some embodiments, one or more portions of the flexible elongate member 104 may be disposable.

In many embodiments, the transducer set 110 may include one or more sensors, transmitters, receivers, transceivers, imagers, and/or lights to facilitate effective monitoring and/or control of the flexible elongate member 104 and/or its environment (e.g., conditions, aspects, or characteristics of a cannulation procedure). For example, transducer set 110 may include one or more of an optical sensor, a laser, an ultrasonic transceiver, a distance sensor (e.g., range finder), a pressure sensor, a localization sensor, an electromagnetic sensor, a capacitive sensor, an inductive sensor, fiber optics, a pH sensor, an ultraviolet sensor, an infrared sensor, a spectrometer, a temperature sensor, an accelerometer, a gyroscope, and the like, or combinations thereof.

In various embodiments, transducers in transducer set 110 may monitor the configuration of the flexible elongate member 104 for disturbances, movements, increases in pressure, and the like to compensate and maintain a target configuration. In some embodiments, optical images may be used in conjunction with image analysis to maintain a distal end of the flexible elongate member 104 at a certain distance from a wall of a body lumen, such as to maintain a consistent field of view. In one or more embodiments, transducers in transducer set 110 utilize a rangefinder and/or fiber optics to maintain a certain distance away from a wall of a body lumen or characteristic thereof (e.g., papilla).

In several embodiments, data may be exchanged with an operator via user interface 116. For example, feedback from the transducer set 110 may be communicated to an operator via user interface 116, such as via one or more of lights, sounds, vibration, or other means to guide the operator. Further, data output via the user interface 116 may be adjusted or customized based on input received via user interface 116. In some embodiments, user interface 116 may include a screen for viewing and/or a control mechanism (e.g., a joystick, touchscreen, keyboard, or the like). In many embodiments, user interface 116 may enable an operator to identify the optimal location and/or position for the flexible elongate member 104. In many such embodiments, this may occur prior to and/or separately for visualization during a procedure, such as after cannulation and tenting.

In various embodiments, the positional control set 108 may include one or more components configured to adjust at least a portion of the flexible elongate member 104. For example, positional control set 108 may include one or more of a motor, a vibrator, a powered joint, a manual joint, an articulation joint, a telescopic joint, a balloon, a stabilizer, a pitch joint, a yaw joint, a roll joint, an actuator, a tract, a wheel, an arm, and the like, or combinations thereof. For example, positional control set 108 may include a motor, a joint, a balloon, and a stabilizer. In embodiments, positional control set 108 may include a plurality of motors, or joints, or balloons, or stabilizers.

More generally, in various embodiments, a set (e.g., positional control set 108, transducer set 110) may refer to one or more components, or combinations thereof, with at least one common characteristics. In some embodiments, one or more aspects of the positional control set 108 and the transducer set 110 may overlap. For instance, a transducer may be included in a positional control. In various embodiments, a transducer may be disposed on or in a positional control. For example, a pressure sensor may be disposed on or within a balloon used for positional control. In such examples, device manager 106 may inflate/deflate the balloon based on data from the pressure sensor to adjust the flexible elongate member 104. In many embodiments, positional controls in positional control set 108 may be used by device manager 106 to respond to disturbances captured by sensors in transducer set 110. In many such embodiments, the response to disturbances may be overridden and/or controlled based on input received via user interface 116.

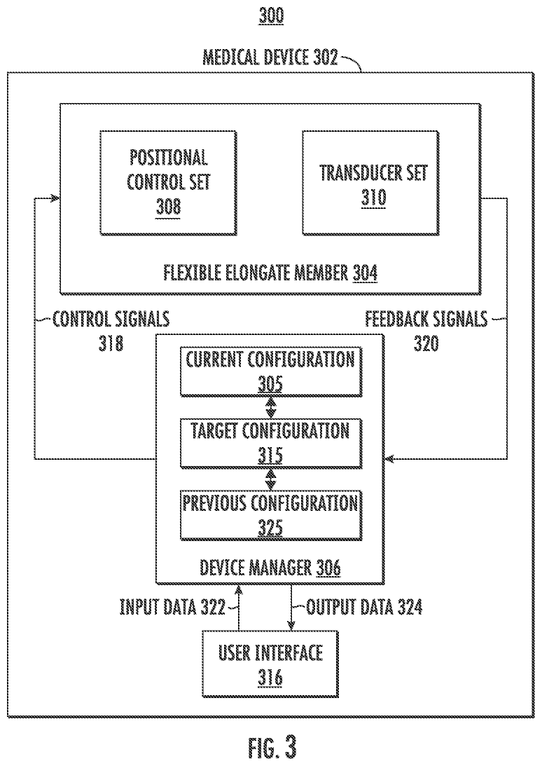

In several embodiments, the medical device 102 may provide guidance prior to and/or during cannulation of a body lumen, such as the duodenal or biliary duct. As will be described in more detail below, such as with respect to FIG. 3, in one or more embodiments, the medical device 102 may include enhanced monitoring and/or positioning capabilities to improve cannulation procedures. In various embodiments, one or more aspects described herein may be integrated with tools in one or more working channels or integrated into the design of a scope (e.g., a duodenoscope).

In some embodiments, intraoperative guidance using techniques and/or devices described herein, such as during an ERCP procedure, can improve duct cannulation, which can improve both patient outcomes and medical professional training. The ability to monitor, guide, and/or control the configuration of flexible elongate member 104 can better assist a medical professional in achieving a desired configuration/alignment/position. Improving the ease of cannulation procedures, such as the duct cannulation and navigation to the target location in the biliary duct, can decrease procedural time and increase a medical professional's proficiency in performing the procedure. Making the process easier may also reduce the number of inadvertent pancreatic duct cannulations and, as a result, lower pancreatitis rates.

FIG. 2 illustrates an example of an operating environment 200 for flexible elongate member 204 according to one or more embodiments described herein. In some embodiments, environment 200 may include one or more components that are the same or similar to one or more other components described herein. For example, a distal end of flexible elongate member 204 may be the same or similar to distal end of the flexible elongate member 104. In environment 200, flexible elongate member 204 may include a proximal portion 204-1 with a proximal end and a distal portion 204-2 with a distal end. In the illustrated embodiments, the distal portion 204-2 of the flexible elongate member 204 is disposed within a body lumen 226 and includes positional controls 208-1, 208-2 and transducers 210-1, 210-2. Embodiments are not limited in this context.

In one or more embodiments described herein, positional controls 208-1, 208-2 may be utilized to move flexible elongate member 204, such as by contacting a wall 228 of the body lumen 226, based on data from transducers 210-1, 210-2. For instance, device manager 106 may determine a configuration of flexible elongate member 204 based on data generated by transducers 210-1, 210-2. In such instances, device manager 106 may utilize positional controls 208-1, 208-2 to adjust flexible elongate member 204 within the body lumen 226 based on the data generate by transducers 210-1, 210-2. As will be appreciated, transducers and positional controls may be configured any number of ways to enable effective or efficient monitoring/positioning of a flexible elongate member without departing from the scope of this disclosure. Further, body lumen 226 may include any body cavity suitable for a cannulation procedure without departing from the scope of this disclosure.

FIG. 3 illustrates an example of a process flow 300 for medical device 302 according to one or more embodiments described herein. In some embodiments, process flow 300 may include or utilize one or more components that are the same or similar to one or more other components described herein. For example, device manager 306 may be the same or similar to device manager 106. In such examples, they may be similar in the sense that user interface 316 is separate from device manager 306 as opposed to user interface 116 being included in device manager 106. In environment 300, medical device 302 may include flexible elongate member 304, device manager 306, and user interface 316. In the illustrated embodiment, device manager 306 may exchange control signals 318 and feedback signals 320 with flexible elongate member 304 as well as input data 322 and output data 324 with user interface 316. Embodiments are not limited in this context.

In one or more embodiments, device manager 306 may utilize feedback signals 320 to determine a current configuration 305 of the flexible elongate member 304. For example, feedback signals 320 may include data generated by one or more transducers in transducer set 310 that is indicative of a configuration of flexible elongate member 304. In some embodiments, one or more of the feedback signals 320 may be generated in response to one or more of the control signals 318. In several embodiments, one or more of control signals 318 may be used to configure one or more components of positional control set 308 and/or transducer set 310. For example, one or more of control signals 318 may be used to calibrate a transducer in transducer set 310 or a positional control in positional control set 308.

In many embodiments, device manager 306 may communicate the current configuration 305 of the flexible elongate member 304 by generating and communicating output data 324 to user interface 316. In various embodiments, device manager 306 may receive input data 322 via user interface 316 to control one or more aspects of medical device 302. In some embodiments, device manager 306 may determine the target configuration 315 based on input data 322. In several embodiments, device manager 306 may generate one or more of control signals 318 based on input data 322. For example, device manager 306 may generate one or more of control signals 318 to operate positional control set 308 to achieve or maintain flexible elongate member 304 in the target configuration 315 determined based on input data 322.

In several embodiments, device manager 306 may provide guidance via user interface 316 and/or adjust flexible elongate member 304 via positional control set 308 based on comparison of the current configuration 305 to a target configuration 315. For instance, device manager 306 may provide guidance by generating output data 324 to cause user interface 316 to display the current and target configurations 305, 315 of flexible elongate member 304. In an additional, or alternative embodiment, device manager 306 may adjust flexible elongate member 304 by generating one or more control signals 318 to operate one or more positional controls in positional control set 308. In many embodiments, device manager 306 may determine the target configuration 315 based on one or more of a previous configuration 325 of the flexible elongate member 304 and input data 322 received via user interface 316.

In one or more embodiments, user interface 316 may enable an operator, such as a medical processional, to change a position, update a target configuration 315, and/or override automated movements of flexible elongate member 304. In various embodiments, such as after cannulation and/or tenting, the user interface 316 may be used to adjust the location of the flexible elongate member 304 to optimize visualization. For example, user interface 316 may be used to adjust the location of the flexible elongate member 304 to optimize visualization once inside a biliary duct. In several embodiments, the flexible elongate member 304 may be locked into position, such as based on input data 322 received via user interface 316. In several such embodiments, this may allow an operator to focus on a cannulation procedure and/or manipulating tools in a working channel. Further, positional disturbances created by manipulating tools in the working channel may be automatically corrected to maintain a configuration and/or visualization.

In various embodiments, device manager 306 may take input from transducer set 310 via feedback signals 320 generated within a body lumen, such as a duodenum. In many embodiments, the feedback signals 320 may include at least one signal providing positional information of the flexible elongate member 304. In some embodiments, the positional information may include a location within the duodenum, such as with respect to the intestinal wall, relative to the rotation of the flexible elongate member 304 and/or an angle of the distal end. Positional information may be provided for various regions/locations along the length of the flexible elongate member 304. For instance, positional information may be provided from the distal end of the flexible elongate member 304 and/or at predefined locations along the scope (e.g., where transducers/positional controls are disposed).

In some embodiments, transducer set 310 may include a force sensor, or similar, to generate feedback signals to enable device manager 306 to protect patient anatomies, such as by ensuring excessive pressure is not applied to the anatomy. In various embodiments, force sensors could be incorporated at the distal end and/or along the length of the flexible elongate member 304 to monitor and adjust the orientation of the flexible elongate member 304 so as excessive pressure on tissue is minimized.

In several embodiments, control over flexible elongate member 304 may be manual, automatic, and/or semiautomatic. In some embodiments, manual control may enable an operator to align the trajectory/path to navigate to the desired location, such as in the duodenum. In some such embodiments, manual control may include the ability of an operator to lock in coordinates/view/location once the flexible elongate member 304 is in an appropriate position for one or more aspects of a cannulation procedure. In various embodiments, automatic control may allow an operator to identify where in the anatomy to navigate, such as based on pre-procedure scans. In various such embodiments, the medical device 302 may then automatically navigate the flexible elongate member 304 to the location and lock the flexible elongate member in place. In one or more embodiments, semiautomatic control may primarily utilize manual control with minor adjustments handled automatically. For instance, minor adjustments to relieve pressure exerted by the flexible elongate member 304 on the wall of a body lumen may be automatically carried out.

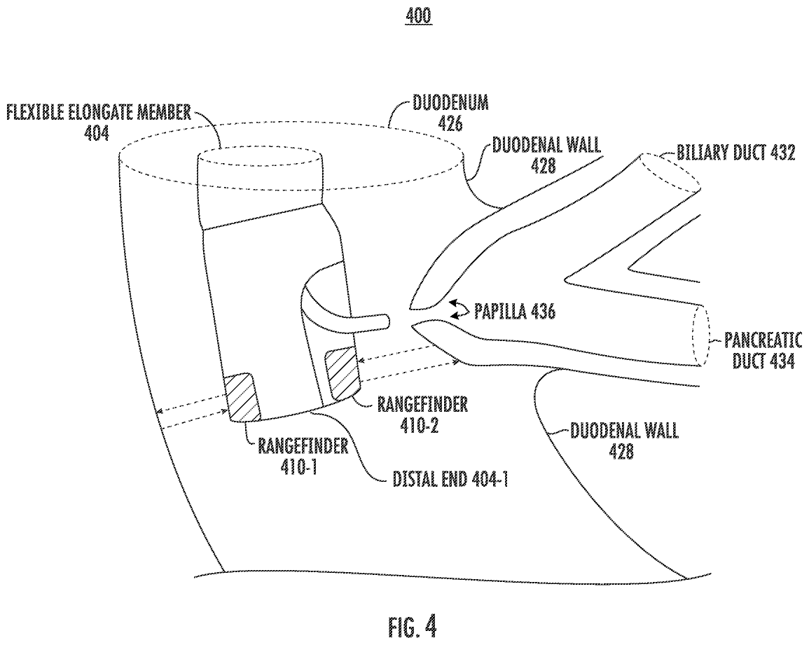

FIG. 4 illustrates exemplary aspects of positioning flexible elongate member 404 in environment 400 according to one or more embodiments described herein. In some embodiments, environment 400 may include or utilize one or more components that are the same or similar to one or more other components described herein. For example, flexible elongate member 404 may be the same or similar to flexible elongate member 104. Environment 400 may include a distal end of flexible elongate member 404 with distal end 404-1 positionable within duodenum 426. In the illustrated embodiments, duodenum 426 may include a duodenal wall 428 with a characteristic comprising a papilla 436 that includes the common entryway to a biliary duct 432 and a pancreatic duct 434. Additionally, distal end of the flexible elongate member 404 may include rangefinders 410-1, 410-2. In some embodiments, a flexible elongate member may additionally, or alternatively, include one or more other positional sensors. In one or more embodiments described herein, rangefinders 410-1, 410-2 may be utilized to determine a position and or configuration of the distal end 404-1 of flexible elongate member 404. For instance, feedback signals generated by rangefinders 410-1, 410-2 may be used to determine the distal end 404-1 position relative to the duodenal wall 428. In such instances, the distance relative to duodenal wall 428 may be used to monitor changes in position during a cannulation procedure (e.g., ERCP procedure). Embodiments are not limited in this context.

In several embodiments, the rangefinders 410 may be located at or proximate the distal end 404-1. In many embodiments, the rangefinders 410 may be located on opposite sides of the flexible elongate member. In various embodiments, one or more positional sensors may be disposed circumferentially around a flexible elongate member. In some embodiments, one or more positional sensors may be disposed along the length of a flexible elongate member.

In the illustrated embodiment, rangefinder 410-1 may determine a distance between a first portion of the flexible elongate member 404 and a first portion of the duodenal wall 428 and rangefinder 410-2 may determine a distance between a second portion of the flexible elongate member 404 and a second portion of the duodenal wall 428. In one or more embodiments described herein, a variety of types and/or combinations of positional sensors may be disposed about the flexible elongate member 404 in a configuration to enable measuring a position/condition/environment of one or more portions/components. In some embodiments, a flexible elongate member may include an optical sensor and/or a light source. In some such embodiments, the optical sensor and/or light source may be utilized to verify or communicate sensor data, such as data from rangefinders 410.

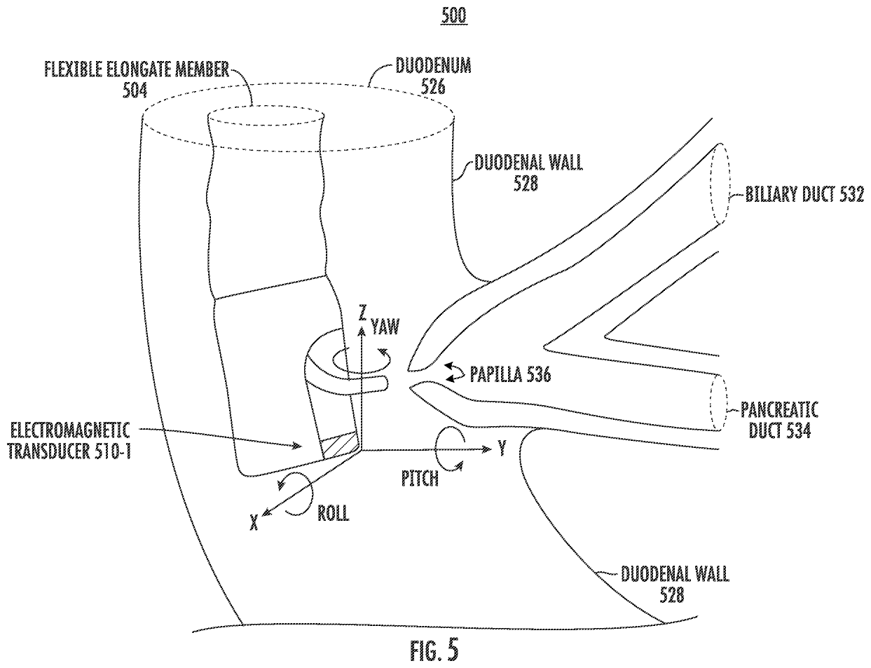

FIG. 5 illustrates exemplary aspects of positioning flexible elongate member 504 in environment 500 according to one or more embodiments described herein. In some embodiments, environment 500 may include or utilize one or more components that are the same or similar to one or more other components described herein. For example, flexible elongate member 504 may be the same or similar to flexible elongate member 104. Environment 500 may include distal end of flexible elongate member 504 positionable within duodenum 526. In the illustrated embodiments, duodenum 526 may include a duodenal wall 528 comprising a papilla 536 that includes the common entryway to a biliary duct 532 and a pancreatic duct 534. Additionally, flexible elongate member 504 may include electromagnetic transducer 510-1. In one or more embodiments described herein, electromagnetic transducer 510-1 may be utilized to determine a position and or configuration of the distal end of flexible elongate member 504. For instance, feedback signals generated by electromagnetic transducer 510-1 may be used to track coordinates and rotational axes of the flexible elongate member 504 within a body lumen and/or maintain the set of coordinates and rotational axes. Embodiments are not limited in this context.

In many embodiments, the electromagnetic transducer 510-1 may be used to determine the location/position/orientation/configuration of the distal end of the flexible elongate member 504. In many such embodiments, the location/position of the distal end of the flexible elongate member 504 may be determined as a set of cartesian coordinates (e.g., x, y, and z). In various embodiments, the orientation of the distal end of the flexible elongate member 504 may be determined as a set of rotational coordinates. For example, rotation about the z-axis may refer to yaw 552, rotation about the y-axis may refer to pitch 554, and rotation about the x-axis may refer to roll 556. In some embodiments, configuration, location, position, and orientation may be used interchangeably.

In several embodiments, a plurality of transducers (e.g., electromagnetic transducer 510-1) may be disposed along the flexible elongate member 504 to determine the location/position/orientation of the flexible elongate member 504. In some embodiments, each transducer may measure one or more attributes of the location/orientation of the portion of the flexible elongate member 504 proximate the transducer. In many embodiments, the attributes measured by each of the transducers may be combined, such as by device manager 106 to determine the location/position/orientation of multiple portions of the flexible elongate member relative to each other. For example, the attributes measured by each of the transducers may be combined by device manager 106 to determine the location and orientation of the entire flexible elongate member 504.

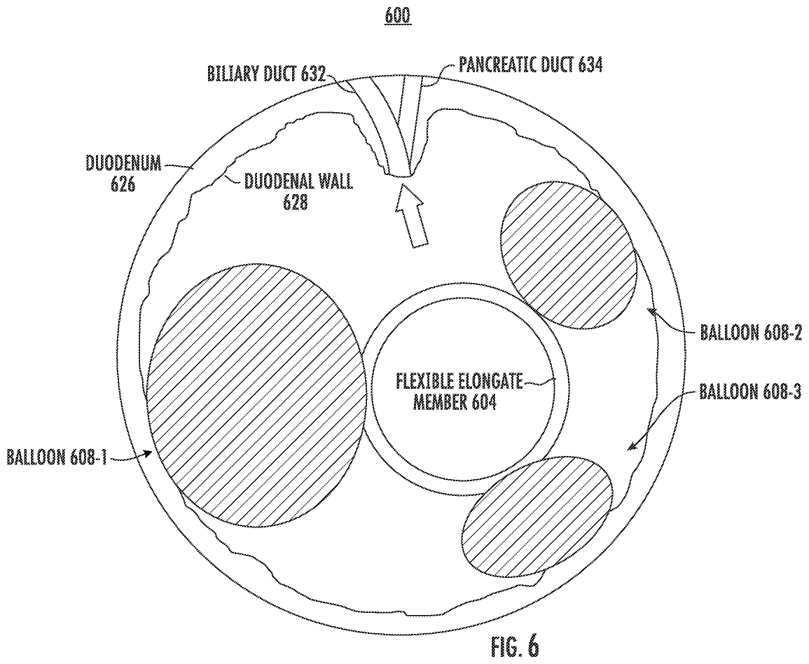

FIG. 6 illustrates exemplary aspects of positioning flexible elongate member 604 in environment 600 according to one or more embodiments described herein. In some embodiments, environment 600 may include or utilize one or more components that are the same or similar to one or more other components described herein. For example, flexible elongate member 604 may be the same or similar to flexible elongate member 104. Environment 600 may include a cross-sectional view of flexible elongate member 604 disposed within duodenum 626. In the illustrated embodiments, duodenum 626 may include a duodenal wall 628 with a characteristic comprising a common entrance point to a biliary duct 632 and a pancreatic duct 634. Additionally, flexible elongate member 604 may include balloons 608-1, 608-2, 608-3. In one or more embodiments described herein, environment 600 may illustrate an example of holding flexible elongate member 604 in place using several positional controls (e.g., balloons 608-1, 608-2, 608-3) contacting the duodenum wall 628. In one or more such embodiments, balloons 608-1, 608-2, 608-3 may comprise positional controls that are operated (e.g., inflated/deflated) to position flexible elongate member 604 within duodenum 626. Embodiments are not limited in this context.

In several embodiments, a target position within duodenum 626, such as to gain access to the biliary duct 632, may be achieved or maintained by inflating/deflating balloons 608-1, 608-2, 608-3. In some embodiments, each of balloons 608-1, 608-2, 608-3 may include a pressure sensing transducer used to inflate/deflate the balloons 608-1, 608-2, 608-3 as needed to position or maintain a position of the flexible elongate member 604 within duodenum 626. In many embodiments, balloons 608-1, 608-2, 608-3 may be independently controlled (e.g., by device manager 106 based on feedback signals) to position the flexible elongate member 604. For example, forces acting on the flexible elongate member 604 may be responded to by increasing pressure within one or more of balloons 608-1, 608-2, 608-3 to counter dynamic back pressures exerted on anatomy, such as back pressures varying based on a guidewire angle, deflection, or position. In various embodiments, independent control of balloons 608-1, 608-2, 608-3 may enable off-center positioning within the duodenum 626. In many embodiments, the balloons 608-1, 608-2, 608-3 may be positioned to avoid contact near the entrance point to the biliary duct 632 (e.g., papilla). In many such embodiments, avoiding contact with the entryway to the biliary duct 632 can reduce irritation, limiting adverse outcomes.

In some embodiments, the flexible elongate member 604 may include one or more transducers to measure attributes of the balloons 608. For instance, a transducer may be included for each balloon 608 to measure the internal pressure of a respective balloon. In several embodiments, the balloons 608 may be attached to the flexible elongate member 604. In various embodiments, one or more portions of the balloons 608 may retract into the flexible elongate member when deflated. In the illustrated embodiments, a distance between balloons 608-1, 608-2 may be larger than the distance between balloons 608-1, 608-3 and balloons 608-2, 608-3 to prevent the balloons from interfering with gaining access to a selected anatomy, such as biliary duct 632. In embodiments, any number of balloons can be used. For example, a single balloon with a plurality of segments that can be independently inflated/deflated may be used. In such example, the balloon may include an opening for receiving the flexible elongate member 604.

FIG. 7 illustrates an embodiment of an exemplary computing architecture 700 that may be suitable for implementing various embodiments as previously described. In various embodiments, the computing architecture 700 may comprise or be implemented as part of an electronic device and/or medical device. In some embodiments, the computing architecture 700 may be representative, for example, of one or more components described herein. In some embodiments, computing architecture 700 may be representative, for example, of a computing device that implements or utilizes one or more portions of components and/or techniques described herein, such as medical device 102, device manager 106, one or more of positional control set 108, one or more of transducer set 110, elongate member interface 112, circuitry 114, and/or user interface 116. The embodiments are not limited in this context.

As used in this application, the terms “system” and “component” and “module” can refer to a computer-related entity, either hardware, a combination of hardware and software, software, or software in execution, examples of which are provided by the exemplary computing architecture 700. For example, a component can be, but is not limited to being, a process running on a processor, a processor, a hard disk drive, multiple storage drives (of optical and/or magnetic storage medium), an object, an executable, a thread of execution, a program, and/or a computer. By way of illustration, both an application running on device manager 106 and the device manager 106 can be a component. One or more components can reside within a process and/or thread of execution, and a component can be localized on one computer and/or distributed between two or more computers. Further, components may be communicatively coupled to each other by various types of communications media to coordinate operations. The coordination may involve the uni-directional or bi-directional exchange of information. For instance, the components may communicate information in the form of signals communicated over the communications media. The information can be implemented as signals allocated to various signal lines. In such allocations, each message is a signal. Further embodiments, however, may alternatively employ data messages. Such data messages may be sent across various connections. Exemplary connections include parallel interfaces, serial interfaces, and bus interfaces.

The computing architecture 700 includes various common computing elements, such as one or more processors, multi-core processors, co-processors, memory units, chipsets, controllers, peripherals, interfaces, oscillators, timing devices, video cards, audio cards, multimedia input/output (I/O) components, power supplies, and so forth. The embodiments, however, are not limited to implementation by the computing architecture 700.

As shown in FIG. 7, the computing architecture 700 comprises a processing unit 704, a system memory 706 and a system bus 708. The processing unit 704 can be any of various commercially available processors, including without limitation an AMD® Athlon®, Duron® and Opteron® processors; ARM® application, embedded and secure processors; IBM® and Motorola® DragonBall® and PowerPC® processors; IBM and Sony® Cell processors; Intel® Celeron®, Core (2) Duo®, Itanium®, Pentium®, Xeon®, and XScale® processors; and similar processors. Dual microprocessors, multi-core processors, and other multi-processor architectures may also be employed as the processing unit 704.

The system bus 708 provides an interface for system components including, but not limited to, the system memory 706 to the processing unit 704. The system bus 708 can be any of several types of bus structure that may further interconnect to a memory bus (with or without a memory controller), a peripheral bus, and a local bus using any of a variety of commercially available bus architectures. Interface adapters may connect to the system bus 708 via a slot architecture. Example slot architectures may include without limitation Accelerated Graphics Port (AGP), Card Bus, (Extended) Industry Standard Architecture ((E)ISA), Micro Channel Architecture (MCA), NuBus, Peripheral Component Interconnect (Extended) (PCI(X)), PCI Express, Personal Computer Memory Card International Association (PCMCIA), and the like.

The system memory 706 may include various types of computer-readable storage media in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory (e.g., one or more flash arrays), polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (e.g., USB memory, solid state drives (SSD) and any other type of storage media suitable for storing information. In the illustrated embodiment shown in FIG. 7, the system memory 706 can include non-volatile memory 710 and/or volatile memory 712. In some embodiments, system memory 706 may include main memory. A basic input/output system (BIOS) can be stored in the non-volatile memory 710.

The computer 702 may include various types of computer-readable storage media in the form of one or more lower speed memory units, including an internal (or external) hard disk drive (HDD) 714, a magnetic floppy disk drive (FDD) 716 to read from or write to a removable magnetic disk 718, and an optical disk drive 720 to read from or write to a removable optical disk 722 (e.g., a CD-ROM or DVD). The HDD 714, FDD 716 and optical disk drive 720 can be connected to the system bus 708 by an HDD interface 724, an FDD interface 726 and an optical drive interface 728, respectively. The HDD interface 724 for external drive implementations can include at least one or both of Universal Serial Bus (USB) and Institute of Electrical and Electronics Engineers (IEEE) 994 interface technologies. In various embodiments, these types of memory may not be included in main memory or system memory.

The drives and associated computer-readable media provide volatile and/or nonvolatile storage of data, data structures, computer-executable instructions, and so forth. For example, a number of program modules can be stored in the drives and memory units 710, 712, including an operating system 730, one or more application programs 732, other program modules 734, and program data 736. In one embodiment, the one or more application programs 732, other program modules 734, and program data 736 can include or implement, for example, the various techniques, applications, and/or components described herein.

A user can enter commands and information into the computer 702 through one or more wire/wireless input devices, for example, a keyboard 738 and a pointing device, such as a mouse 740. Other input devices may include microphones, infra-red (IR) remote controls, radio-frequency (RF) remote controls, game pads, stylus pens, card readers, dongles, finger print readers, gloves, graphics tablets, joysticks, keyboards, retina readers, touch screens (e.g., capacitive, resistive, etc.), trackballs, trackpads, sensors, styluses, and the like. These and other input devices are often connected to the processing unit 704 through an input device interface 742 that is coupled to the system bus 708 but can be connected by other interfaces such as a parallel port, IEEE 1394 serial port, a game port, a USB port, an IR interface, and so forth.

A monitor 744 or other type of display device is also connected to the system bus 708 via an interface, such as a video adaptor 746. The monitor 744 may be internal or external to the computer 702. In addition to the monitor 744, a computer typically includes other peripheral output devices, such as speakers, printers, and so forth.

The computer 702 may operate in a networked environment using logical connections via wire and/or wireless communications to one or more remote computers, such as a remote computer 748. In various embodiments, one or more interactions described herein may occur via the networked environment. The remote computer 748 can be a workstation, a server computer, a router, a personal computer, portable computer, microprocessor-based entertainment appliance, a peer device or other common network node, and typically includes many or all of the elements described relative to the computer 702, although, for purposes of brevity, only a memory/storage device 750 is illustrated. The logical connections depicted include wire/wireless connectivity to a local area network (LAN) 752 and/or larger networks, for example, a wide area network (WAN) 754. Such LAN and WAN networking environments are commonplace in offices and companies, and facilitate enterprise-wide computer networks, such as intranets, all of which may connect to a global communications network, for example, the Internet.

When used in a LAN networking environment, the computer 702 is connected to the LAN 752 through a wire and/or wireless communication network interface or adaptor 756. The adaptor 756 can facilitate wire and/or wireless communications to the LAN 752, which may also include a wireless access point disposed thereon for communicating with the wireless functionality of the adaptor 756.

When used in a WAN networking environment, the computer 702 can include a modem 758, or is connected to a communications server on the WAN 754 or has other means for establishing communications over the WAN 754, such as by way of the Internet. The modem 758, which can be internal or external and a wire and/or wireless device, connects to the system bus 708 via the input device interface 742. In a networked environment, program modules depicted relative to the computer 702, or portions thereof, can be stored in the remote memory/storage device 750. It will be appreciated that the network connections shown are exemplary and other means of establishing a communications link between the computers can be used.

The computer 702 is operable to communicate with wire and wireless devices or entities using the IEEE 802 family of standards, such as wireless devices operatively disposed in wireless communication (e.g., IEEE 802.16 over-the-air modulation techniques). This includes at least Wi-Fi (or Wireless Fidelity), WiMax, and Bluetooth™ wireless technologies, among others. Thus, the communication can be a predefined structure as with a conventional network or simply an ad hoc communication between at least two devices. Wi-Fi networks use radio technologies called IEEE 802.11x (a, b, g, n, etc.) to provide secure, reliable, fast wireless connectivity. A Wi-Fi network can be used to connect computers to each other, to the Internet, and to wire networks (which use IEEE 802.3-related media and functions).

Various embodiments may be implemented using hardware elements, software elements, or a combination of both. Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores” may be stored on a tangible, machine readable medium and supplied to various customers or manufacturing facilities to load into the fabrication machines that actually make the logic or processor. Some embodiments may be implemented, for example, using a machine-readable medium or article which may store an instruction or a set of instructions that, if executed by a machine, may cause the machine to perform a method and/or operation in accordance with the embodiments. Such a machine may include, for example, any suitable processing platform, computing platform, computing device, processing device, computing system, processing system, computer, processor, or the like, and may be implemented using any suitable combination of hardware and/or software. The machine-readable medium or article may include, for example, any suitable type of memory unit, memory device, memory article, memory medium, storage device, storage article, storage medium and/or storage unit, for example, memory, removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, and the like, implemented using any suitable high-level, low-level, object-oriented, visual, compiled and/or interpreted programming language.

The medical devices of the present disclosure are not limited to duodenoscopes, and may include a variety of medical devices for accessing body passageways, including, for example, catheters, ureteroscopes, bronchoscopes, colonoscopes, arthroscopes, cystoscopes, hysteroscopes, and the like.

All of the devices and/or methods disclosed and claimed herein can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

What is claimed is:

1. A medical device, comprising:
a flexible elongate member having a proximal end, a distal end, and a length therebetween;
a set of one or more positional controls disposed along the length of the flexible elongate member, each positional control in the set of one or more positional controls configured to adjust at least a portion of the flexible elongate member; and
a set of one or more transducers disposed along the length of the flexible elongate member, at least a portion of the set of one or more transducers configured to generate data indicative of a configuration of the flexible elongate member, wherein at least one positional control in the set of positional controls is configured for operation by circuitry comprised in a device manager based on data indicative of the configuration of the flexible elongate member generated by the set of one or more transducers.

2. The medical device of claim 1, comprising the device manager, the circuitry of the device manager configured to monitor a position of the flexible elongate member within a body lumen based on the data generated by the set of one or more transducers.

3. The medical device of claim 1, comprising the device manager and a user interface communicatively coupled to the device manager, the circuitry of the device manager configured to operate at least one positional control in the set of one or more positional controls based on input received via the user interface.

4. The medical device of claim 1, comprising the device manager and a user interface communicatively coupled to the device manager, the circuitry of the device manager configured to display the configuration of the flexible elongate member via the user interface.

5. The medical device of claim 1, the set of one or more positional controls comprising a joint, an actuator, a motor, a balloon, a guidewire, or combinations thereof.

6. The medical device of claim 1, comprising the device manager, the circuitry of the device manager configured to determine a suggested position of at least a portion of the flexible elongate member.

7. The medical device of claim 6, comprising the device manager and a user interface communicatively coupled to the device manager, the circuitry of the device manager configured to communicate the suggested position of at least a portion of the flexible elongate member via the user interface.

8. An apparatus, comprising:
a processor; and
memory comprising instructions that when executed by the processor cause the processor to:
identify one or more feedback signals generated by a set of one or more transducers disposed along a length of a flexible elongate member;
determine a current configuration of at least one portion of the flexible elongate member within a body lumen based on one or more portions of the one or more feedback signals generated by the set of one or more transducers;
compare the current configuration of the at least one portion of the flexible elongate member to a target configuration of the at least one portion of the flexible elongate member; and
generate one or more control signals to operate at least one positional control in a set of one or more positional controls disposed along the length of the flexible elongate member to adjust the flexible elongate based on comparison of the current configuration of the at least one portion of the flexible elongate member to the target configuration.

9. The apparatus of claim 8, the memory comprising instructions that when executed by the processor cause the processor to communicate the target configuration of the at least one portion of the flexible elongate member via a user interface.

10. The apparatus of claim 8, the memory comprising instructions that when executed by the processor cause the processor to communicate the current configuration of the at least one portion of the flexible elongate member via a user interface.

11. The apparatus of claim 8, the memory comprising instructions that when executed by the processor cause the processor to determine an environment of the at least one portion of the flexible elongate member within the body lumen based on at least one portion of the one or more feedback signals generated by the set of one or more transducers.

12. The apparatus of claim 8, the memory comprising instructions that when executed by the processor cause the processor to operate at least one positional control in the set of one or more positional controls based on input received via a user interface.

13. The apparatus of claim 8, the memory comprising instructions that when executed by the processor cause the processor to determine the target configuration of the at least one portion of the flexible elongate member based on a previous configuration of the at least one portion of the flexible elongate member.

14. The apparatus of claim 8, the memory comprising instructions that when executed by the processor cause the processor to determine the target configuration of the at least one portion of the flexible elongate member based on input received via a user interface.

15. A method, comprising:
identifying one or more feedback signals generated by a set of one or more transducers disposed along a length of a flexible elongate member;
determining a current configuration of at least one portion of the flexible elongate member within a body lumen based on one or more portions of the one or more feedback signals generated by the set of one or more transducers;
comparing the current configuration of the at least one portion of the flexible elongate member to a target configuration of the at least one portion of the flexible elongate member; and
generating one or more control signals to operate at least one positional control in a set of one or more positional controls disposed along the length of the flexible elongate member to adjust the flexible elongate based on comparison of the current configuration of the at least one portion of the flexible elongate member to the target configuration.

16. The method of claim 15, comprising communicating the target configuration of the at least one portion of the flexible elongate member via a user interface.

17. The method of claim 15, comprising communicating the current configuration of the at least one portion of the flexible elongate member via a user interface.

18. The method of claim 15, comprising determining an environment of the at least one portion of the flexible elongate member within the body lumen based on at least one portion of the one or more feedback signals generated by the set of one or more transducers.

19. The method of claim 15, comprising operating at least one positional control in the set of one or more positional controls based on input received via a user interface.

20. The method of claim 15, comprising determining the target configuration of the at least one portion of the flexible elongate member based on a previous configuration of the at least one portion of the flexible elongate member.
